(19) United States
(12) Patent Application Publication
(10) Pub. No.: US 2019/0101984 A1
(43) Pub. Date: Apr. 4, 2019

(54) HEARTRATE MONITOR FOR AR WEARABLES

(71) Applicant: Facebook Technologies, LLC, Menlo Park, CA (US)

(72) Inventors: Sharvil Shailesh Talati, Seattle, WA (US); Nicholas Daniel Trail, Bothell, WA (US)

(21) Appl. No.: 15/720,945

(22) Filed: Sep. 29, 2017

(51) Int. Cl. G06F 3/01; G06F 3/0481; G06N 99/00; A61B 5/024

(52) U.S. Cl. CPC G06F 3/015; G06F 3/013; A61B 5/02427; G06N 99/005; A61B 5/02438; G06F 3/04812

(57) ABSTRACT

A heartrate monitor distributed adjusted reality system for producing rendered environments includes an eyewear device and a neckband formed from a first arm, second arm, and computation compartment. Optical, electrical and visual sensors are distributed across the neckband and eyewear device at points of contact with a user's tissue. Machine learning modules combine optical, electrical and visual measurements produced by the optical, electrical and visual sensors to determine a user's heartrate and/or other vitals. The neckband and eyewear device may be capable of adapting an adjusted reality environment in response to a determine user heartrate or other vitals.

### BACKGROUND

This application generally relates to heartrate monitors, and specifically relates to heartrate monitors and biometric monitors embedded in wearable augmented reality (AR), mixed reality (MR) and/or virtual reality (VR) systems.

Wearable adjusted reality systems and environments allow a user to directly or indirectly view a real world environment augmented by generated sensory input, which may be super-imposed on the real world environment. Sensory input can be any form of media, such as sound, video, graphics, etc. The wearable adjusted reality device provides an immersive environment for the user, capable of dynamically responding to a user's interaction with the adjusted reality environment. Ideally, an adjusted reality system would seamlessly integrate into a user's interactions and perceptions of the world, while allowing the world they view to adapt to fit the user. Moreover, monitoring a user's physical state during his/her immersion in an adjusted reality environment may be an important metric for adapting the adjusted reality environment to the user. However, conventional adjusted reality systems do not track a user's physical state.

### SUMMARY

A heartrate monitor distributed system is configured to integrate heartrate monitoring into a plurality of devices that together provide a virtual reality (VR), augmented reality (AR) and/or mixed reality (MR) environment. The system includes a neckband that provides a surface over which electrical and/or optical sensing may measure a user's heartrate. The neckband also handles processing offloaded to it from other devices in the system. The system includes an eyewear device communicatively coupled with the neckband. At least one of the neckband and the eyewear device measures an electrical signal associated with a user's heart activity. A light source is optically coupled to a light detector. The light source and light detector are located on at least one of the neckband and the eyewear device, and measure an optical signal associated with the user's heart activity. A controller is configured to determine a heartrate of the user based on the electrical signal and the optical signal measured at the eyewear device and/or the neckband.

In some embodiments, visual information of a user's eye may also be collected and used with the electrical and optical signals to determine a user's heartrate. In some embodiments, machine learning modules may use a combination of visual information, electrical signals and optical signals to generate vitals models that map these measured signals to a user's heartrate and/or other vitals. Distributing heartrate monitoring functions across the eyewear device and neckband increase the number of contact sites with a user's tissue at which these measurements can be made. Additionally, offloading power, computation and additional features from devices in the system to the neckband device reduces weight, heat profile and form factor of those devices. Integrating heartrate monitoring in an adjusted reality environment allows the augmented environments to better adapt to a user.

### BRIEF DESCRIPTION OF THE DRAWINGS

FIG. 1 is a diagram of a heartrate monitor distributed system, in accordance with an embodiment.

FIG. 2 is a perspective view of a user wearing the heartrate monitor distributed system, in accordance with an embodiment.

FIG. 3A is a first overhead view of a user wearing the heartrate monitor distributed system, in accordance with an embodiment.

FIG. 3B is a second overhead view of a user wearing the heartrate monitor distributed system, in accordance with an embodiment.

FIG. 4 is an overhead view of a system for measuring an optical signal associated with a user's heart activity, in accordance with an embodiment.

FIG. 5 is a side view of a system for measuring an optical signal associated with a user's heart activity, in accordance with an embodiment.

FIG. 6 is example data of optical data and electrical data associated with a user's heart activity, in accordance with an embodiment.

FIG. 7A is a block diagram of a first machine learning module for determining a user's vitals, in accordance with an embodiment.

FIG. 7B is a block diagram of a second machine learning module for determining a user's vitals, in accordance with an embodiment.

FIG. 8 is a block diagram of a heart rate monitor distributed system, in accordance with an embodiment.

### DETAILED DESCRIPTION OF THE DRAWINGS

AR and/or mixed reality (MR) devices allow a user to directly or indirectly view a real world environment augmented by generated sensory input, such as sound, video, graphics, etc. The generated sensory input may be super-imposed on the real world environment, allowing the user to interact with both simultaneously, or may be completely immersive such that the environment is entirely generated. Augmented and virtual environments typically rely on generated media that is visual and/or audio-based. And because of this many AR, MR and/or VR devices, collectively referred to as adjusted reality devices, attach to a user's head, where they may be closer to a user's ears for audio media and display images in a user's field of view for visual media.

Ideally, adjusted reality devices dynamically adapt to a user, providing environments that reflect the user's needs. Measuring a user's vitals is an important indication of a user's physical state, providing information about stress level, sleep cycles, activity intensity, fitness and health. Knowing a user's vitals may allow the augmented reality environment to adjust to a user. For example, if a user is running through an adjusted reality environment, the environment could adapt to reflect the intensity of the user's workout as measured by a heartrate monitor. In other examples, a user's emotional state may be detected through measurement of a user's vitals, and the adjusted reality device may adapt content in response. The prevalence of wearable devices for health and fitness tracking also indicates considerable user interest in accessing his or her own health data in real time, which gives the user the ability to adjust activity based on feedback and metrics provided by these trackers.

Most existing heart rate monitors determine a user's heartrate based on either electrical or optical sensors. Electrical signals detect electrical potential changes in the skin and tissue that result from the heart muscle's electrophysiologic pattern of depolarizing and repolarizing over the course of each heartbeat. Optical signals detect changes in light absorption that result from the distension of the arteries, capillaries and arterioles and corresponding change in tissue volume over the course of each heartbeat. Electrical signals are typically measured from a user's chest, where the potential difference is more easily detected due to proximity to the heart. Optical signals are typically measured from thin, easily illuminated segments of a user's body, such as a finger, with good blood flow characteristics. Because electrical sensing and optical sensing are conducted at different locations of the body, and can achieve the necessary accuracy at these locations, heartrate monitors are typically dedicated to a single sensing method.

To integrate heartrate monitors with an adjusted reality device, the existing heart rate monitor technology thus depends on additional dedicated devices located on a user's chest or hand, which may be inconvenient for the user. The present invention moves heartrate monitoring to a distributed adjusted reality device located on a user's head. To mitigate any reduction in accuracy, in some examples the present invention combines both optical and electrical sensing to determine a user's heartrate. In some examples, optical sensing may be conducted without electrical sensing. In some examples, electrical sensing may be conducted without optical sensing. The present invention also includes a machine learning module that trains measured optical and electrical signals against known vitals, such as heartrate, to improve accuracy of the heartrate monitor. A user's heartrate and/or other vital sign, such as pulse, blood pressure, respiration rate, blood-oxygen level, etc. are collectively referred herein as a user's vitals.

Embodiments of the invention may include or be implemented in conjunction with an artificial reality system. Artificial reality is a form of reality that has been adjusted in some manner before presentation to a user, which may include, e.g., a virtual reality (VR), an augmented reality (AR), a mixed reality (MR), a hybrid reality, or some combination and/or derivatives thereof. Artificial reality content may include completely generated content or generated content combined with captured (e.g., real-world) content. The artificial reality content may include video, audio, haptic feedback, or some combination thereof, and any of which may be presented in a single channel or in multiple channels (such as stereo video that produces a three-dimensional effect to the viewer). Additionally, in some embodiments, artificial reality may also be associated with applications, products, accessories, services, or some combination thereof, that are used to, e.g., create content in an artificial reality and/or are otherwise used in (e.g., perform activities in) an artificial reality. The artificial reality system that provides the artificial reality content may be implemented on various platforms, including a head-mounted display (HMD) connected to a host computer system, a standalone HMD, a mobile device or computing system, or any other hardware platform capable of providing artificial reality content to one or more viewer.

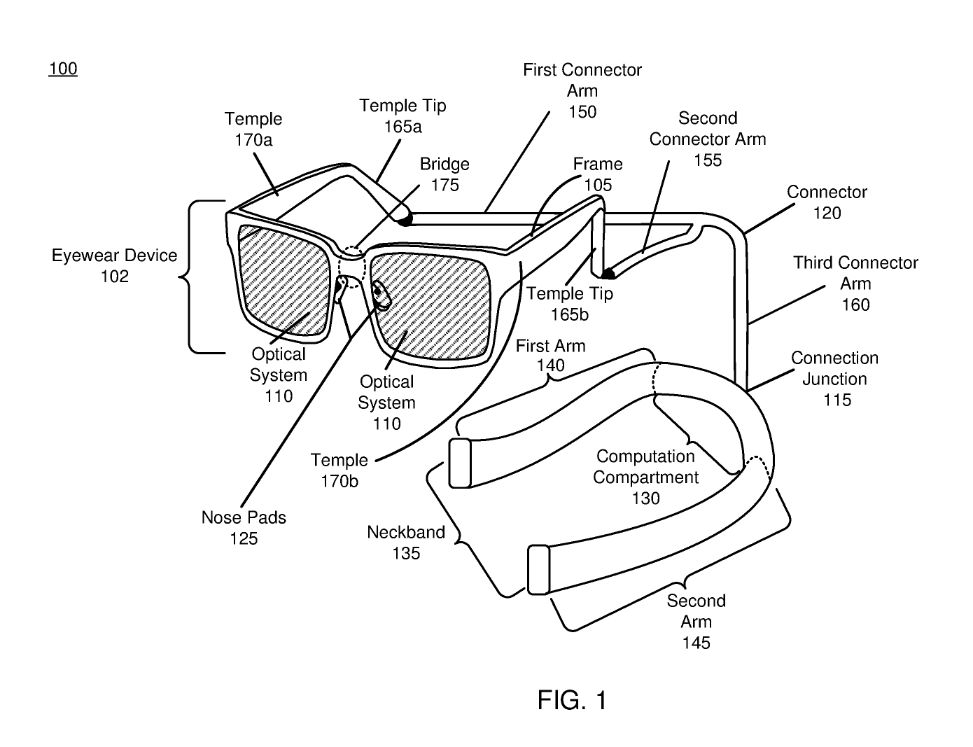

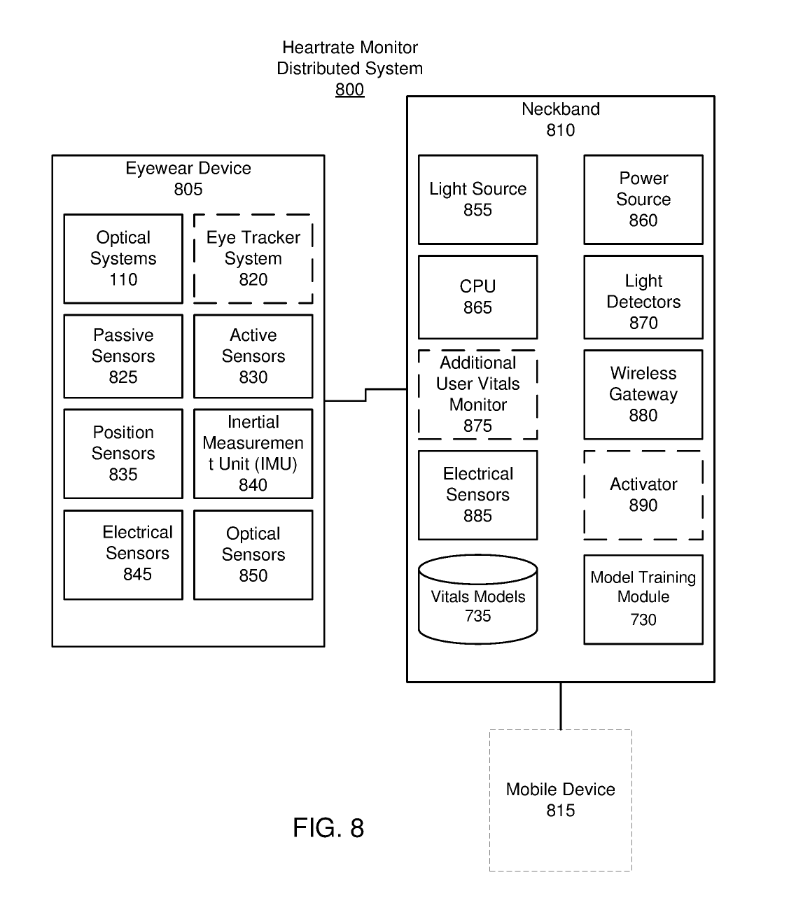

FIG. 1 is a diagram of a heartrate monitor distributed system 100, in accordance with an embodiment. The heartrate monitor distributed system 100 includes an eyewear device 102 and a neckband 135. A heartrate monitor may be integrated into the eyewear device 102, neckband 135, or both. In alternate embodiments, the distributed system 100 may include additional components (e.g., a mobile device as discussed in detail below with regard to FIG. 8).

The eyewear device 102 provides content to a user of the distributed system 100, as well as contact points with a user's head and tissue for heartrate and vitals sensing. The eyewear device 102 includes two optical systems 110. The eyewear device 102 may also include a variety of sensors other than the heartrate and vitals sensors, such as one or more passive sensors, one or more active sensors, one or more audio devices, an eye tracker system, a camera, an inertial measurement unit (not shown), or some combination thereof. As shown in FIG. 1, the eyewear device 102 and optical systems 110 are formed in the shape of eyeglasses, with the two optical systems 110 acting as eyeglass “lenses” and a frame 105. The frame 105 includes temples 170a and 170b, and temple tips 165a and 165b, which rest on the side of a user's face and are secured behind a user's ears. The frame 105 is attached to a connector 120 at temple tips 165a and 165b. The connector junction 115 attaches connector 120 to the neckband 135.

The eyewear device 102 provides several contact points with a user's head and tissue for heartrate and vitals sensing. If a user's heartrate is detected through electrical sensing, the heartrate monitor distributed system 100 detects a potential difference between two electrical sensors, such as electrodes. Thus for electrical sensing, there must be at least two contact points with the user on the device. In some examples, the two contact points measure an electrical signal across the same tissue region and the distance between the two contact points is small. If a user's heartrate is detected through optical sensing, the optical sensor may measure light transmitted through a user's tissue (transmitted measurement) using at least two contact points, or may illuminate a section of a user's tissue and measure the reflected light (reflected measurement), using only one contact point. Any of the contact points described herein may be used for either single-contact, optical reflected measurement, or as one contact in either an optical transmitted measurement or an electrical measurement using at least a second contact point.

The eyewear device 102 sits on a user's head as a pair of eyeglasses. Nose pads 125 are contact points with a user's nose, and provide a contact surface with a user's tissue through which an electrical or optical signal could be measured. Bridge 175 connecting the optical systems 110 rests on the top of the user's nose. The weight of eyewear device 102 may be partially distributed between the nose pads 125 and bridge 175. The weight of the eyewear device 102 may ensure that the contact points at the nose pads 125 and bridge 175 remain stationary and secure for electrical or optical measurement. Temples 170a and 170b may be contact points with the side of a user's face. Temple tips 165a and 165b curve around the back of a user's ear, and may provide contact points with the user's ear tissue through which an electrical or optical signal could be measured.

Optical systems 110 present visual media to a user. Each of the optical systems 110 may include a display assembly. In some embodiments, when the eyewear device 102 is configured as an AR eyewear device, the display assembly also allows and/or directs light from a local area surrounding the eyewear device 102 to an eyebox (i.e., a region in space that would be occupied by a user's eye). The optical systems 110 may include corrective lenses, which may be customizable for a user's eyeglasses prescription. The optical systems 110 may be bifocal corrective lenses. The optical systems 110 may be trifocal corrective lenses.

The display assembly of the optical systems 110 may be composed of one or more materials (e.g., plastic, glass, etc.) with one or more refractive indices that effectively minimize the weight and widen a field of view of the eyewear device 102 visual system. In alternate configurations, the eyewear device 102 includes one or more elements between the display assembly and the eye. The elements may act to, e.g., correct aberrations in image light emitted from the display assembly, correct aberrations for any light source due to the user's visual prescription needs, magnify image light, perform some other optical adjustment of image light emitted from the display assembly, or some combination thereof. An element may include an aperture, a Fresnel lens, a convex lens, a concave lens, a liquid crystal lens, a liquid or other deformable surface lens, a diffractive element, a waveguide, a filter, a polarizer, a diffuser, a fiber taper, one or more reflective surfaces, a polarizing reflective surface, a birefringent element, or any other suitable optical element that affects image light emitted from the display assembly.

Examples of media presented by the eyewear device 102 include one or more images, text, video, audio, or some combination thereof. The eyewear device 102 can be configured to operate, in the visual domain, as a VR Near Eye Device (NED), an AR NED, an MR NED, or some combination thereof. For example, in some embodiments, the eyewear device 102 may augment views of a physical, real-world environment with computer-generated elements (e.g., images, video, sound, etc.). The eyewear device 102 may include a speaker or any other means of conveying audio to a user, such as bone conduction, cartilage conduction, open-air or in-ear speaker, etc.

The visual and/or audio media presented to the user by the eyewear device 102 may be adjusted based on the user's vitals detected by the distributed system 100. For example, in response to detecting a user's elevated heartrate due to stress or anxiety, the visual and/or audio media could be adjusted to provide soothing sounds and relaxing images, or to limit ancillary information which may not be pertinent to a user's task. In another example, the audio and/or visual media could increase or decrease in intensity to reflect the user's degree of exertion during a workout, as indicated by a detected heartrate. In another example, the audio and/or visual media could provide white noise if a user's heartrate indicated he/she was sleeping.

In other embodiments, the eyewear device 102 does not present media or information to a user. For example, the eyewear device 102 may be used in conjunction with a separate display, such as a coupled mobile device or laptop (not shown). In other embodiments, the eyewear device 102 may be used for various research purposes, training applications, biometrics applications (e.g., fatigue or stress detection), automotive applications, communications systems for the disabled, or any other application in which heartrate and vitals detection can be used.

The eyewear device 102 may include embedded sensors (not shown) in addition to the heartrate and vitals sensors, such as 1-dimensional (1D), 2-dimensional (2D) imagers, or scanners for localization and stabilization of the eyewear device 102, as well as sensors for understanding the user's intent and attention through time. The sensors located on the eyewear device 102 may be used for Simultaneous Localization and Mapping (SLAM) calculations, which may be carried out in whole or in part by the processor embedded in the computation compartment 130 and/or a processor located in a coupled mobile device, as described in further detail with reference to FIG. 8. Embedded sensors located on the eyewear device 102 may have associated processing and computation capabilities.

In some embodiments, the eyewear device 102 further includes an eye tracking system (not shown) for tracking a position of one or both eyes of a user. Note that information about the position of the eye also includes information about an orientation of the eye, i.e., information about a user's eye-gaze. The eye tracking system may include a camera, such as a red, green, and blue (RGB) camera, a monochrome, an infrared camera, etc.

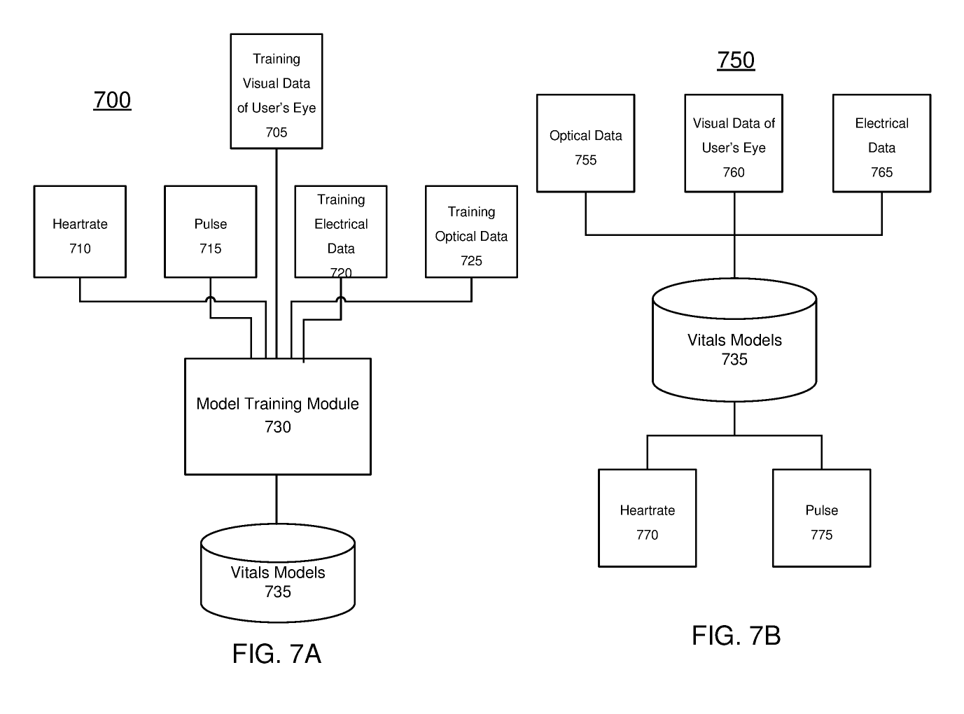

The camera used in the eye tracking system may also be used to detect a user's vitals by providing visual data of a user's eye. The camera may provide images and video of a user's eye movement, orientation, color and/or that of the surrounding eye tissue. By amplifying and magnifying otherwise imperceptible motions of a user's eye or surrounding eye tissue, one may be able to detect a user's heartrate and/or vitals. This amplification may be done by decomposing images and/or video through an Eulerian video magnification, a spatial decomposition technique described in the following paper: Hao-Yu Wu and Michael Rubinstein and Eugene Shih and John Guttag and Frédo Durand and William T. Freeman. “Eulerian Video Magnification for Revealing Subtle Changes in the World.” ACM Trans. Graph. (Proceedings SIGGRAPH 2012), vol. 31, no. 4, 2012. The visual data of a user's eye may then be provided to a machine learning module, as described in further detail with reference to FIG. 7A and 7B. By amplifying changes in the visual data of a user's eye, the machine learning module can detect changes in color, eye movement, eye orientation, and/or any other characteristic of an eye that results from a user's pulse. For example, the skin surrounding a user's eye may change color as a result of blood being periodically circulated to the user's skin tissue. An increase in red tones, followed by a decrease in red tones may correspond to the systole and diastole phases of the cardiac cycle. By detecting these periodic changes in color, the user's heartrate and other vital information such as blood pressure may be determined by the machine learning module.

Amplifying changes in the visual data of a user's eye may also reveal periodic motion in the user's eye tissue or surrounding skin tissue that results from blood being circulated to the tissue. For example, blood vessels may expand and contract as a result of the increase and decrease in blood pressure during the systole and diastole phases of the cardiac cycle, respectively. This periodic expansion and contraction may allow for the measurement of a user's heartrate and/or other vitals. Thus by amplifying motion in visual data of the user's eye, the user's heartrate and other vital information such as blood pressure may be determined by the machine learning module.

In addition to collecting visual data of the user's eye, the camera in the eye tracking system may track the position and orientation of the user's eye. Based on the determined and tracked position and orientation of the eye, the eyewear device 102 adjusts image light emitted from one or both of the display assemblies. In some embodiments, the eyewear device 102 adjusts focus of the image light through the optical systems 110 and ensures that the image light is in focus at the determined angle of eye-gaze in order to mitigate the vergence-accommodation conflict (VAC). Additionally or alternatively, the eyewear device 102 adjusts resolution of the image light by performing foveated rendering of the image light, based on the position of the eye. Additionally or alternatively, the eyewear device 102 uses the information regarding a gaze position and orientation to provide contextual awareness for the user's attention, whether on real or virtual content. The eye tracker generally includes an illumination source and an imaging device (camera). In some embodiments, components of the eye tracker are integrated into the display assembly. In alternate embodiments, components of the eye tracker are integrated into the frame 105. Additional details regarding incorporation of eye tracking system and eyewear devices may be found at, e.g., U.S. patent application Ser. No. 15/644,203, which is hereby incorporated by reference in its entirety.

Computation for the eye-tracking system, amplifying visual data of the user's eye, and the machine learning module may be carried out by the processor located in the computation compartment 130 and/or a coupled mobile device, as described in further detail with reference to FIG. 8. The eyewear device 102 may include an Inertial Measurement Unit (IMU) sensor (not shown) to determine the position of the eyewear device relative to a user's environment, as well as detect user movement. The IMU sensor may also determine the relative spatial relationship between the eyewear device 102 and the neckband 135, which may provide information about the position of the user's head relative to the position of the user's body. Here the neckband 135 may also include an IMU sensor (not shown) to facilitate alignment and orientation of the neckband 135 relative to the eyewear device 102. The IMU sensor on the neckband 135 may determine the orientation of the neckband 135 when it operates independently of the eyewear device 102. The eyewear device 102 may also include a depth camera assembly (not shown), which may be a Time-of-Flight (TOF) camera, a Structured Light (SL) camera, a passive and/or active stereo system, and may include an infrared (IR) light source and detection camera (not shown). The eyewear device 102 may include a variety of passive sensors, such as a Red, Green, and Blue (RGB) color camera, passive locator sensors, etc. The eyewear device 102 may include a variety of active sensors, such as structured light sensors, active locators, etc. The number of active sensors may be minimized to reduce overall weight, power consumption and heat generation on the eyewear device 102. Active and passive sensors, as well as camera systems may be placed anywhere on the eyewear device 102.

The neckband 135 is a wearable device that provides additional contact points with a user's tissue for determining the heartrate and other vitals of the user. The neckband 135 also performs processing for intensive operations offloaded to it from other devices (e.g., the eyewear device 102, a mobile device, etc.). The neckband 135 is composed of a first arm 140 and a second arm 145. As shown, a computation compartment 130 is connected to both the first arm 140 and the second arm 145. The computation compartment 130 is also attached to the connector 120 by connector junction 115. The connector 120 attaches the computation compartment 130 to the frame 105 of the eyewear device 102 at the temple tips 165a and 165b.

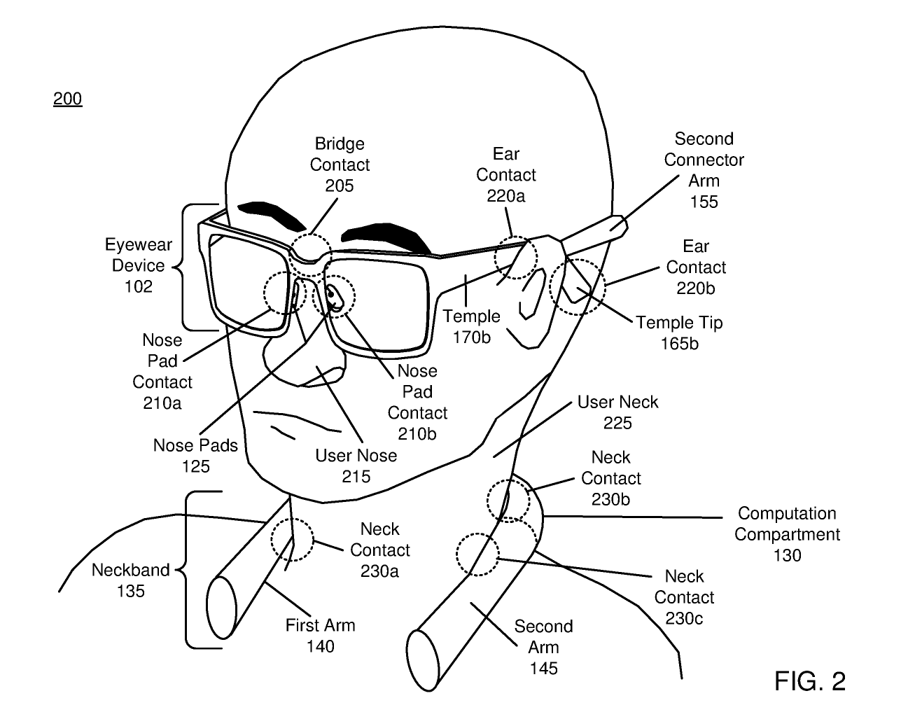

The neckband 135, composed of the first arm 140, the second arm 145 and the computation compartment 130, is formed in a “U” shape that conforms to the user's neck and provides a surface in contact with the user's neck through which a user's heartrate and other vitals may be measured. The neckband 135 is worn around a user's neck, while the eyewear device 102 is worn on the user's head as described in further detail with respect to FIGS. 2-5. The first arm 140 and second arm 145 of the neckband 135 may each rest on the top of a user's shoulders close to his or her neck such that the weight of the first arm 140 and second arm 145 are carried by the user's neck base and shoulders. The computation compartment 130 may sit on the back of a user's neck. The connector 120 is long enough to allow the eyewear device 102 to be worn on a user's head while the neckband 135 rests around the user's neck. The connector 120 may be adjustable, allowing each user to customize the length of connector 120.

The neckband 135 provides a surface in contact with a user's neck tissue over which a user's heartrate and vitals may be sensed. This sensing surface may be the interior surface of the neckband 135. If a user's heartrate is detected through electrical sensing, the heartrate monitor distributed system 100 detects a potential difference between two electrical sensors, such as electrodes. Thus for electrical sensing, there must be at least two contact points with the user on the neckband 135. If a user's heartrate is detected through optical sensing, the optical sensor may measure light transmitted through a user's tissue (transmitted measurement) using at least two contact points on the neckband 135, or may illuminate a section of a user's tissue and measure the reflected light (reflected measurement), using only one contact point on the neckband 135. In some examples, the optical sensor illuminates a section of a user's tissue and measures the reflected light (reflected measurement) using more than one contact point on the neckband 135.

Because the neckband provides a large surface over which to measure a user's heartrate of vital, electrical signals may be measured between several electrodes located at multiple points on the neckband 135. Electrical signals may be measured between electrodes located on the first arm 140, computation compartment 130, and/or second arm 145, or any combination thereof. Electrical signals may also be measured between electrodes located on the same sub-section of the neckband 135. The electrical signals may be processed by a processor located in the computation compartment 130. Electrical sensors may be powered by a battery compartment located on the neckband 135 (not shown). The electrical signals measured by electrical sensors located on the neckband 135 may be provided to a machine learning module as training electrical data, or input electrical data for determining a user's vitals, as described in further detail with reference to FIG. 7A and FIG. 7B.

The neckband 135 may also include optical sensors for determining an optical signal of a user's heartrate and/or other vitals. Because of the large surface area in contact with a user's neck, a number of optical sensors may be placed at several locations on the neckband 135 for either transmitted or reflected measurement. Transmitted measurement may be made between a light source located on the first arm 140, computation compartment 130, or second arm 145, and a light detector located on the first arm 140, computation compartment 130, or second arm 145, or any combination thereof. The lights source and light detector in a transmitted measurement are optically coupled. A single light source may be optically coupled to multiple light detectors distributed across several points on the interior surface of the neckband 135. Multiple light sources may be optically coupled to multiple light detectors distributed across several points on the interior surface of the neckband 135.

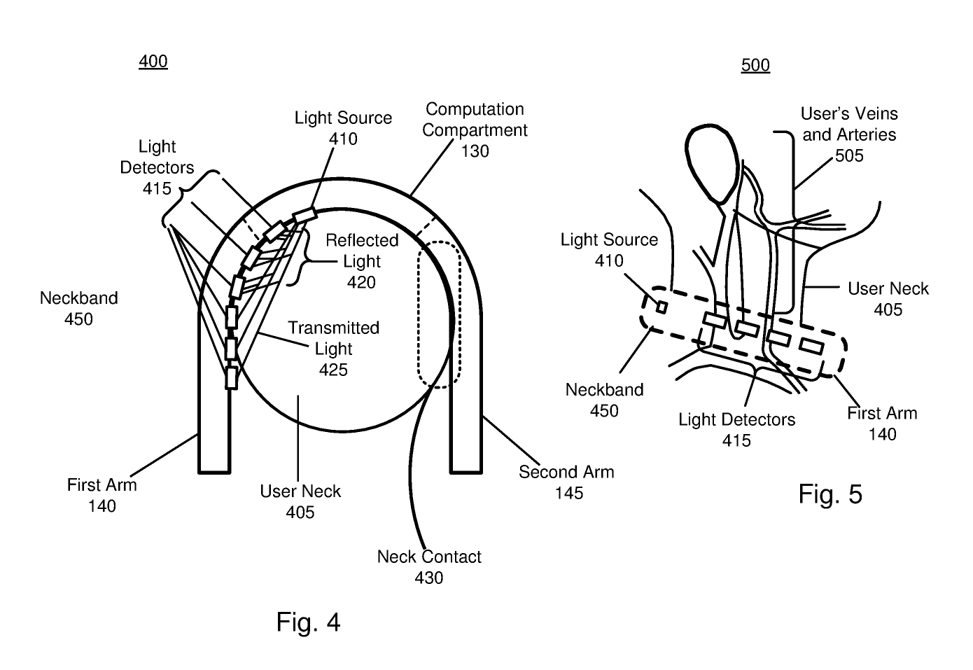

Sensors for reflected optical measurements may be located on the first arm 140, computation compartment 130, and/or second arm 145. Sensors for reflected optical measurements may be located on neckband 135 in addition to sensors for transmitted optical measurements, such that neckband 135 measures both a transmitted and reflected optical signal of a user's vitals. The optical signals may be processed by a processor located in the computation compartment 130. Optical sensors may be powered by a battery compartment located on the neckband 135 (not shown). The optical signals measured by optical sensors located on the neckband 135 may be provided to the machine learning module as training optical data, or input optical data for determining a user's vitals, as described in further detail with reference to FIG. 7A and FIG. 7B. Configurations of the placement of optical sensors on the neckband 135 are shown in further detail with reference to FIG. 4 and FIG. 5.

The neckband 135 may include both optical sensors and electrical sensors, such that neckband 135 measures both an optical signal and an electrical signal of a user's vitals.

In some embodiments, the computation compartment 130 houses a processor (not shown), which processes information generated by any of the sensors or camera systems on the eyewear device 102 and/or the neckband 135. The processor located in computation compartment 130 may include the machine learning module, as discussed in further detail with reference to FIG. 7A and FIG. 7B. Information generated by the eyewear device 102 and the neckband 135 may also be processed by a mobile device, such as the mobile device described in further detail with reference to FIG. 8. The processor in the computation compartment 130 may process information generated by both the eyewear device 102 and the neckband 135, such as optical and electrical measurements of the user's heartrate and other vitals. The connector 120 conveys information between the eyewear device 102 and the neckband 135, and between the eyewear device 102 and the processor in the computation compartment 130. In some examples, the first arm 140, and second arm 145 may also each have an embedded processor (not shown). In these examples, the connector 120 conveys information between the eyewear device 102 and the processor in each of the first arm 140, the second arm 145 and the computation compartment 130. The information may be in the form of optical data, electrical data, or any other transmittable data form. Moving the processing of information generated by the eyewear device 102 to the neckband 135 reduces the weight and heat generation of the eyewear device 102, making it more comfortable to the user.

The processor embedded in the computation compartment 130 and/or one or more processors located elsewhere in the system 100 process information. For example, the processor may compute all calculations to determine a user's vitals; compute all machine learning calculations associated with a machine learning module shown in FIG. 7A and FIG. 7B; compute some or all inertial and spatial calculations from the IMU sensor located on the eyewear device 102; compute some or all calculations from the active sensors, passive sensors, and camera systems located on the eyewear device 102; perform some or all computations from information provided by any sensor located on the eyewear device 102; perform some or all computation from information provided by any sensor located on the eyewear device 102 in conjunction with a processor located on a coupled external device, such as a mobile device as described in further detail with reference to FIG. 8; or some combination thereof.

In some embodiments, the neckband 135 houses the power sources for any element on the eyewear device 102, and one or more sensors located on the neckband 135. The power source may be located in a battery compartment, which may be embedded in the first arm 140, second arm 145, computation compartment 130, or any other sub-assembly of the neckband 135. The power source may be batteries, which may be re-chargeable. The power source may be lithium ion batteries, lithium-polymer battery, primary lithium batteries, alkaline batteries, or any other form of power storage. The computation compartment 130 may have its own power source (not shown) and/or may be powered by a power source located on the neckband 135. Locating the power source for the heartrate monitor distributed system 100 on the neckband 135 distributes the weight and heat generated by a battery compartment from the eyewear device 102 to the neckband 135, which may better diffuse and disperse heat, and also utilizes the carrying capacity of a user's neck base and shoulders. Locating the power source, computation compartment 130 and any number of other sensors on the neckband 135 may also better regulate the heat exposure of each of these elements, as positioning them next to a user's neck may protect them from solar and environmental heat sources.

The neckband 135 may include a multifunction compartment (not shown). The multifunction compartment may be a customizable compartment in which additional feature units may be inserted and removed by a user. Additional features may be selected and customized by the user upon purchase of the neckband 135. Additional features located in the multifunction compartment may provide additional information regarding the user's vitals, and/or may provide information to the machine learning module to determine a user's heartrate. For example, the multifunction compartment may include a pedometer, which may determine a user's pace, calories burned, etc. The multifunction compartment may also include an alert when irregular heartrate activity is detected. Examples of other units that may be included in a multifunction compartment are: a memory unit, a processing unit, a microphone array, a projector, a camera, etc.

The computation compartment 130 is shown as a segment of the neckband 135 in FIG. 1. However, the computation compartment 130 may also be any sub-structures of neckband 135, such as compartments embedded within neckband 135, compartments coupled to sensors embedded in neckband 135, compartments coupled to a multifunction compartment, and may be located anywhere on neckband 135.

Any of the above components may be located in any other part of the neckband 135. There may be any number of power sources distributed across the neckband 135. There may be any number of computation compartments 130 distributed across the neckband 135.

The connector 120 is formed from a first connector arm 150 that is latched to the temple tip 165a of the eyewear device 102. A second connector arm 155 is latched to the temple tip 165b of the eyewear device 102, and forms a “Y” shape with the first connector arm 150 and second connector arm 155. A third connector arm 160 is shown latched to the neckband 135 computation compartment 130 at connector junction 115. The third connector arm 160 may also be latched at the side of the neckband 135, such as along the first arm 140 or second arm 145. The first connector arm 150 and the second connector arm 155 may be the same length so that the eyewear device 102 sits symmetrically on a user's head. The connector 120 conveys both information and power from the neckband 135 to the eyewear device 102. The connector 120 may also include electrical sensors to determine a user's vitals.

An electrical sensor, such as an electrode, may be attached to the inside of the first connector arm 150, such that the electrical sensor makes contact with a user's head when the eyewear device 102 is worn. An electrical sensor may also be attached to the inside of the second connector arm 155, such that the electrical sensor makes contact with a user's head when the eyewear device 102 is worn. The electrical sensors located on the first connector arm 150 and/or second connector arm 155 may measure an electrical potential between the first connector arm 150 and second connector arm 155. The electrical sensors located on the first connector arm 150 and second connector arm 155 may measure an electrical potential between a second electrical sensor located on the eyewear device 102 and the first connector arm 150 and/or second connector arm 155. The electrical sensors located on the first connector arm 150 and/or the second connector arm 150 may measure an electrical potential between either of the connector arms and a second electrode located on the neckband 135.

Because an electrical sensor located on either of the connector arms may measure an electrical potential across a cross-section of the user's head, the electrical signal measured may contain information about both a user's heartrate and a user's brain activity. Information regarding a user's brain activity may be used to determine a user's intended input into the heartrate monitor distributed system 100, such as a “YES” or “NO” input or an “ON” or “OFF” input. Information regarding a user's brain activity may be used for a brain computer interface (BCI) between the user and the heartrate monitor distributed system 100 and/or any device coupled to the heartrate monitor distributed system 100.

In some examples, the connector 120 conveys information from the eyewear device 102 to the neckband 135. Sensors located on the eyewear device 102 may provide the processor embedded in the computation compartment 130 with sensing data, which may be processed by the processor in the computation compartment 130. The computation compartment 130 may convey the results of its computation to the eyewear device 102. For example, if the result of the processor in the computation compartment 130 is a rendered result to be displayed to a user, the computation compartment sends the information through the connector 120 to be displayed on the optical systems 110. In some examples, there may be multiple connectors 120. For example, one connector 120 may convey power, while another connector 120 may convey information.

In some examples, the connector 120 provides power through magnetic induction at the connector junctions 115. In this example, the connector junction 115 may be retention magnets, as well as the connections of the first connector arm 150 to the temple tip 165a and the second connector arm 155 to the temple tip 165b. The connector 120 may also provide power from the neckband 135 to the eyewear device 102 through any conventional power coupling technique. The connector 120 is flexible to allow for independent movement of the eyewear device 102 relative to the neckband 135. The connector 120 may be retractable, or otherwise adjustable to provide the correct length between the near-eye-display and the neckband 135 for each user, since the distance between a user's head and neck may vary.

In some examples, the eyewear device 102 is wirelessly coupled with the neckband 135. In these examples, the processor embedded in the computation compartment 130 receives information from the eyewear device 102 and the sensors and camera assemblies located on the eyewear device 102 through the wireless signal connection, and may transmit information back to the eyewear device 102 through the wireless signal connection. The wireless connection between the eyewear device 102 and the neckband 135 may be through a wireless gateway or directional antenna, located in the first arm 140 and/or second arm 145 and/or on the eyewear device 102. The wireless connection between the eyewear device 102 and the neckband 135 may be a WiFi connection, a Bluetooth connection, or any other wireless connection capable of transmitting and receiving information. The wireless gateway may also connect the eyewear device 102 and/or the neckband 135 to a mobile device, as described in further detail with reference to FIG. 8.

In some examples in which the eyewear device 102 is wirelessly coupled with the neckband 135, the connector 120 may only transmit power between the neckband 135 and the eyewear device 102. Information between the eyewear device 102 and neckband 135 would thus be transmitted wirelessly. In these examples, the connector 120 may be thinner. In some examples in which the eyewear device 102 is wirelessly coupled with the neckband 135, power may be transmitted between the eyewear device 102 and the neckband 135 via wireless power induction. In some examples, there may be a separate battery or power source located in the eyewear device 102. In some examples in which the eyewear device 102 is wirelessly coupled with the neckband 135, the addition of a connector 120 may be optional.

As shown in FIG. 1, the heartrate monitor distributed system 100 includes both an eyewear device 102 and neckband 135, however it is possible for each of these components to be used separately from each other. For example, the heartrate monitor distributed system 100 may include the eyewear device 102 without the neckband 135. In other embodiments, the heartrate monitor distributed system 100 includes the neckband 135 without the eyewear device 102.

The eyewear device 102 and neckband 135 architecture that forms the heartrate monitor distributed system 100 thus allow for the integration of a heartrate monitor into a user's AR, VR and/or MR experience. The multiple points of contact across the neckband 135, eyewear device 102, and connector arms 150 and 155 provide multiple regions from which sensors may be in contact with a user's tissue to collect electrical and/or optical measurements of a user's heartrate.

The eyewear device 102 and neckband 135 architecture also allow the eyewear device 102 to be a small form factor eyewear device, while still maintaining the processing and battery power necessary to provide a full AR, VR and/or MR experience. The neckband 135 allows for additional features to be incorporated that would not otherwise have fit onto the eyewear device 102. In some embodiments, the eyewear device 102 may weigh less than 60 grams (e.g., 50 grams).

FIG. 2 is a perspective view 200 of a user wearing the heartrate monitor distributed system, in accordance with an embodiment. The eyewear device 102 is worn on a user's head, while the neckband 135 is worn around a user's neck 225, as shown in FIG. 2. A first connector arm 150 (not shown) and second connector arm 155 secure the eyewear device 102 to the user's head. The perspective view 200 shows a number of contact points between the heartrate monitor distributed system 100 as shown in FIG. 1 and the user's tissue, at which electrical and/or optical sensors may be placed.

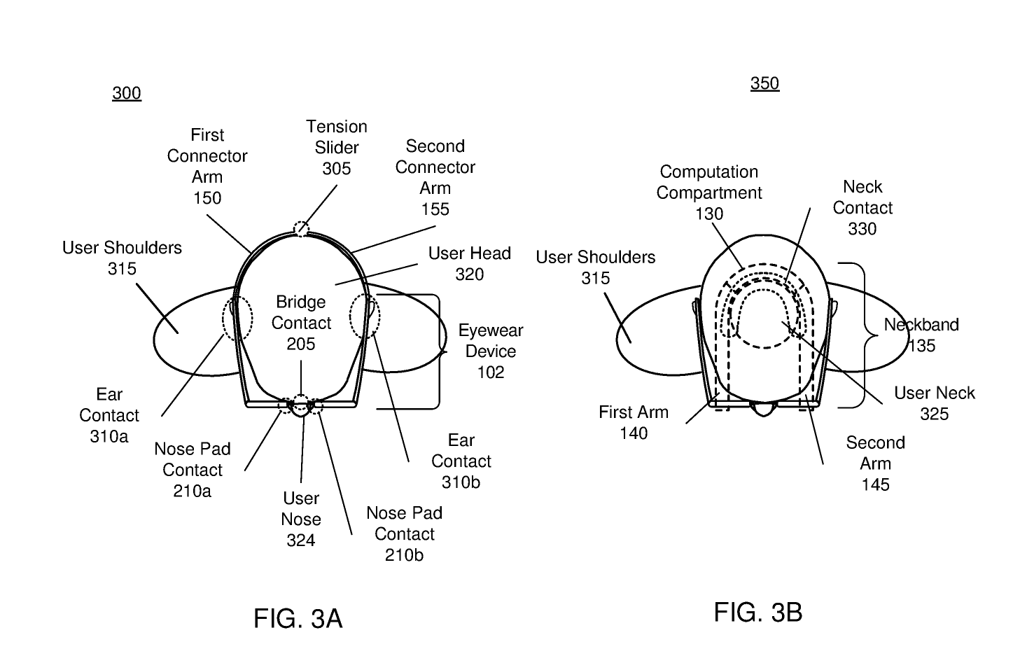

The eyewear device 102 rests on a user's nose 215 on nose pads 125, forming nose pad contacts 210a and 210b. The eyewear device 102 rests on top of a user's nose 215 at bridge contact 205. The temple 170a (not shown) and temple 170b of eyewear device 102 rest against the user's head and ear, as shown at ear contact 220a. The temple tip 165b may also make contact with a user's ear, forming ear contact 220b. The first connector arm 150 (not shown) and second connector arm 155 are secured against the user's head, such that the inner surface of the first connector arm 150 and second connector arm 155 are fully in contact with the user's head. The first connector arm 150 and second connector arm 155 may be additionally secured using a tension slider, as shown in FIG. 3A.

The neckband 135 rests around a user's neck 225 such that the first arm 140 and second arm 145 sit on the tops of the user's shoulders, while the computation compartment 130 rests on the back of the user's neck 225. The first arm 140 makes contact with the user's neck 225 at neck contact 230a, which may be located at the side of the user's neck as shown in FIG. 2. The second arm 145 makes contact with the user's neck 225 at neck contact 230c, which may be located at the side of the user's neck as shown in FIG. 2. The computation compartment 130 makes contact with the user's neck 225 at neck contact 230b, which may be the back of the user's neck 225 as shown in FIG. 2.

At any of the contact points shown in FIG. 2 between the user's tissue and any one of the eyewear device 102, the connector arms 150 and 155, and the neckband 135, an electrical and/or optical sensor may be located. For example, a reflective optical sensor may be located at the ear contact 220b, and produce an optical measurement of the user's vitals. In another example, an electrical sensor may be located at nose pad contact 210b, while a second electrical sensor may be located on the second connector arm 155, and an electrical measurement detected as an electrical potential between the user's nose 215 and the side of the user's head. Any combination of electrical and optical signals may be used at any of the contact points shown in FIG. 2.

Thus as shown in FIG. 2, the eyewear device 102, neckband 135 and connector arms 150 and 155 that form the heartrate monitor distributed system 100 affords a number of different contact points with a user's tissue at which a user's heartrate and/or other vital may be measured.

FIG. 3A is a first overhead view 300 of a user wearing a heartrate monitor distributed system, in accordance with an embodiment. The first overhead view 300 shows the eyewear device 102 in contact with a user's head 320. The first overhead view 300 may be an overhead view of the perspective view 200 as shown in FIG. 2. The eyewear device 102 is the eyewear device 102 as shown in FIG. 1-2.

As shown in FIG. 3A, the eyewear device 102 rests on a user's head 320. The temples 170a and 170b of the eyewear device 102 make contact with the regions around the user's ears at ear contacts 310a and 310b, respectively. The front of the eyewear device 102 contacts the user's head 320 at nose pad contact 210a, nose pad contact 210b, and bridge contact 205. The first connector arm 150 and second connector arm 155 contact the user's head 320 across arcs from the end of the eyewear device 102 to the tension slider 305. In some examples, an electrical potential is measured across a full arc of the user's head, e.g. from the user's nose 324 to the tension slider 305. In some examples, an electrical potential is measured across a fraction of an arc of the user's head, such as between nose pad contact 210a and ear contact 210a, nose pad contact 210b and ear contact 310b, etc. Electrical and/or optical signals measured at any of the contact points shown in first overhead view 300 may be used in a machine learning module as training electrical data, training optical data, input electrical data and/or input optical data, as discussed in further detail with reference to FIG. 7A and 7B.

FIG. 3B is a second overhead view 350 of a user wearing the heartrate monitor distributed system, in accordance with an embodiment. The second overhead view 350 shows the neckband 135 in contact with a user's neck 325. The second overhead view 350 may be an overhead view of the perspective view 200 as shown in FIG. 2. The neckband 135 is the neckband 135 as shown in FIG. 1-2 and may be the neckband 450 as shown in FIG. 4-5 and discussed in further detail below.

As shown in FIG. 3B, the neckband 135 sits on a user's shoulders in direct contact with a user's neck 325. The computation compartment 130 is in contact with the back of the user's neck 325, while the first arm 140 is in contact with the side of the user's neck 325 and the second arm 145 is in contact with the other side of the user's neck 325. As shown in FIG. 3B, the neckband 135 may conform to the shape of the user's neck, providing a contact surface 330 across which electrical and/or optical sensors may be placed to measure a user's vitals. For example, an electrical signal may be measured across the full arc of the neck contact 330. In other examples, an electrical signal is measured across a fraction of the arc of neck contact 330. An example of a configuration of optical sensors across neck contact 330 is discussed in further detail with reference to FIG. 4-5. Electrical and/or optical measurements made across neck contact 330 may be used in a machine learning module as training electrical data, training optical data, input electrical data and/or input optical data, as discussed in further detail with reference to FIG. 7A and 7B.

FIG. 4 is an overhead view of a system 400 for measuring an optical signal associated with a user's heart activity, in accordance with an embodiment. The neckband 450 is in direct contact with the tissue of a user's neck 405, such as across neck contact 430 between the second arm 145 and user neck 405. The neckband 450 includes an arrangement of a light source 410 and light detectors 415 for measuring an optical signal associated with a user's vitals. The neckband 450 may be the neckband 135 as shown in FIG. 1-2 and FIG. 3B.

As shown in FIG. 4, a light source 410 is placed on the inner surface of the computation compartment 130 in contact with a user neck 405. A number of light detectors 415 are optically coupled to the light source 410 and detect both reflected light 420 and transmitted light 425 through the user's neck 405. As shown in FIG. 4, the light source may be optically coupled to the light detectors 415 at an oblique angle, such that the transmitted light 425 is transmitted through a segment of the user neck 405 to a light detector 415 along the first arm 140. The light source 410 may be located on the computation compartment 130 as shown in FIG. 4, or on the first arm 140 or second arm 145. Multiple light sources 410 may be located on any of the first arm 140, computation compartment 130 and/or second arm 145. The light detectors 415 may be located on the first arm 140 as shown in FIG. 4, or may be located on the computation compartment 130 and/or second arm 145. Multiple light detectors may be located on any of the first arm 140, computation compartment 130 and/or second arm 145. Multiple light sources 410 may be optically coupled to multiple light detectors 415.

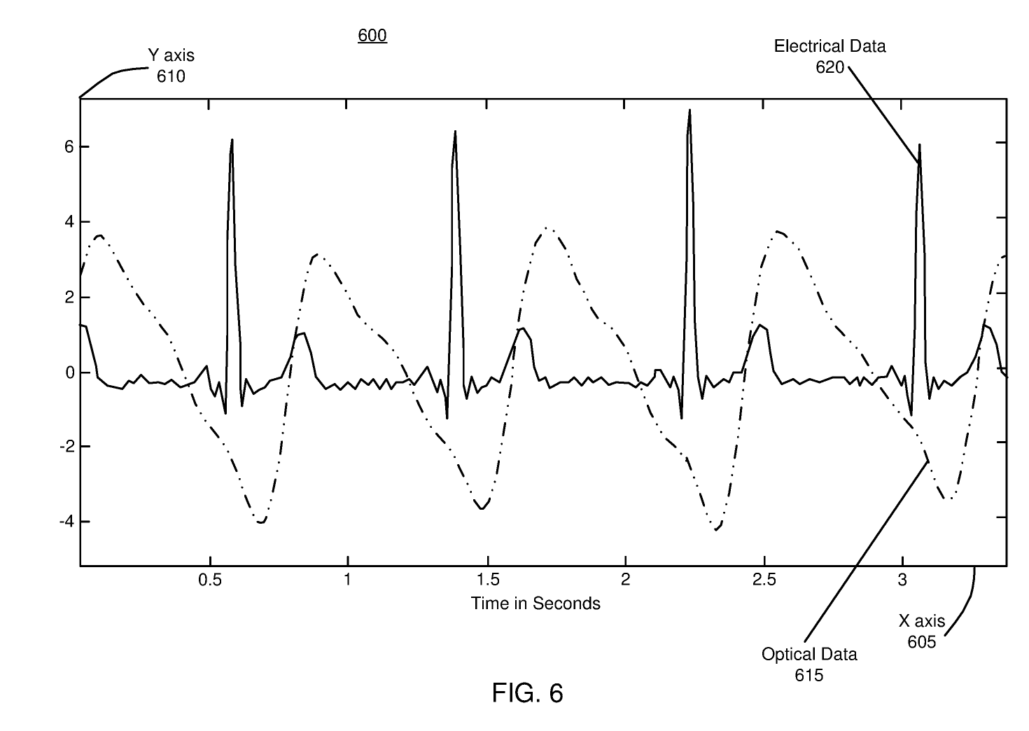

The magnitude of light transmitted from light source 410 may be recorded and measured against the reflected light 420 and transmitted light 425. The optical measurement as shown in FIG. 4 may be a photoplethysmogram (PPG) measurement, whereby changes in the volume of the tissue in the user neck 405 are detected through changes in the absorption of the neck tissue that result from blood being pumped into the skin over the course of a user's cardiac cycle. A Direct Current (DC) signal reflects the bulk absorption properties of a user's skin, while an Alternating Current (AC) component of the signal detected by light detectors 415 reflects absorption changes from the cardiac cycle. An example of the signal detected by light detectors 415 is shown with reference to FIG. 6. In some examples, multiple wavelengths of light are transmitted from multiple light sources, and the signals derived from each wavelength are compared to determine a user's vitals. For example, absorption measurements for different wavelengths may be compared to determine oxygen saturation levels in a user's blood, or a user's pulse rate. In some examples, the different wavelengths may be a red wavelength (620-750 nm) and an infrared wavelength (700 nm-1800 nm). In some examples, the different wavelengths may be a red wavelength, an infrared wavelength, and a green wavelength (495-570 nm).

The light detectors 415 may be any photodetectors or photosensors. The bandwidth of light detectors 415 may be chosen to reflect the bandwidth of the light source 410. Light detectors 415 may include bandpass filters for selecting particular wavelengths of interest out of the reflected light 420 and transmitted light 425. Light source 410 may be any device capable of transmitting light, such as an IR light source, photodiode, Light-emitting Diode (LED), etc. In some examples, light source 410 emits light of wavelengths between 400 nm and 1800 nm. In some examples, light detectors 415 detect light of wavelengths between 400 nm and 1800 nm.

The arrangement of the light source 410 and light detectors 415 as shown in FIG. 4 are an example of transmitted measurement, as discussed with reference to FIG. 1. Thus light is directly transmitted through an arc of tissue, and a measurement of reflected light 420 and transmitted light 425 is made to determine a user's vitals. Alternatively, the light source 410 and light detectors 415 may make a reflective measurement, wherein light is transmitted approximately perpendicularly into tissue of the user's neck 405 and a light detector located close to the light source 410 directly measures only the reflected light. Because the reflected light is fraction of the total transmitted light, the amount of reflected light versus transmitted light can be inferred from the reflected light measurement, rather than directly measuring both reflected light 420 and transmitted light 425 at the light detector 415 as shown in FIG. 4. Any combination of reflected and transmitted optical sensing may be used together.

The neckband 450 thus provides a surface over which transmitted and reflected optical measurements can be made of the tissue of a user's neck 405. Because of the curved form of the neckband 450, light may be transmitted through a segment of a user's neck 405, allowing for a direct measurement of both transmitted light 425 and reflected light 420 at light detectors 415.

FIG. 5 is a side view 500 of a system for measuring an optical signal associated with a user's heart activity, in accordance with an embodiment. Side view 500 shows the neckband 450 as discussed in FIG. 4 being worn on a user neck 405 in proximity to a user's veins and arteries 505. The neckband 450 may be the neckband 135 as shown in FIGS. 1-2 and 3B. As shown in FIG. 5, the first arm 140 has embedded light detectors 415, which are optically coupled to the light source 410. Light detectors 415 and light source 410 are discussed in further detail with reference to FIG. 4. Light may be transmitted through the user neck 405 from light source 410 to light detectors 415, as shown in FIG. 4. As shown in FIG. 5, the proximity of a user's veins and arteries 505 in the user neck 405 to the light source 410 and light detectors 415 make the neckband 450 an ideal location to detect of a user's heartrate and other vitals. The transmitted and reflected light detected by light detectors 415 may pass directly through the user's veins and arteries 505, providing a substantially strong signal of a user's vitals, such as heartrate.

FIG. 6 is example data of optical data 615 and electrical data 620 associated with a user's heart activity, in accordance with an embodiment. The x axis may be in units of time, such as seconds. The y axis may be in units of signal magnitude, such as volts. Electrical data 620 may be a voltage produced as a result of the measurement of a potential difference between two contact points with a user's tissue. Electrical data 620 may be produced by any of the electrical sensors described herein. Optical data 615 may be a voltage measured by a photodetector of reflected and/or transmitted light. Optical data 615 may be produced by any of the optical sensors described herein.

The cardiac cycle produced in both the electrical data 620 and optical data 615 may not be directly measured by any of the electrical and/or optical sensors, but may instead be produced by a machine learning module as a result of a plurality of different measurements. For example, the electrical data 620 may be produced by a machine learning module from a number of different electrical signals, optical signals, and/or visual data of a user's eye. Similarly, the optical data 615 may be produced by a machine learning module from a number of different optical signals, electrical signals, and/or visual data of a user's eye. The machine learning module is described in further detail with reference to FIG. 7A and 7B.

FIG. 7A is a block diagram of a first machine learning module for determining a user's vitals, in accordance with an embodiment. Machine learning module 700 receives a variety of training data to generate vitals models 735. Machine learning module 700 deals with a study of systems that can learn from data they are operating on, rather than follow only explicitly programmed instructions.

As shown in FIG. 7A, vitals models 735 are created through model training module 730 and a variety of training data. The training data consists of a known heartrate 710, known pulse 715, and/or other known user vitals detected by an eyewear device and/or neckband. In some examples, sensors located on the eyewear device and/or neckband produce the training visual data of user's eye 705, training electrical data 720, training optical data 725, and/or other training data. In some examples, additional sensors (not shown) collect training visual data of user's eye 705, training electrical data 720, training optical data 725 and/or other data in addition to the sensors located on the eyewear device and/or neckband. In these examples, the additional sensors may be chest heartrate monitors, pulse oximeters, or any other sensor capable of measuring a user's vitals. Because this data is taken from known vitals, it can be input into a model training module 730 and used to statistically map signals measured by the eyewear device and/or neckband to a user's true vital measurements. The model training module 730 uses machine learning algorithms to create vitals models 735, which mathematically describe this mapping.

The training visual data of a user's eye 705, known heartrate 710, known pulse 715, training electrical data 720, training optical data 725 are very large datasets taken across a wide cross section of people and under a variety of different environmental conditions, such as temperature, sun exposure of the eyewear device and/or neckband, at various battery power levels, etc. The training datasets are large enough to provide a statistically significant mapping from measured signals to true vitals. A range of known heartrates 710, pulse 715, and/or other vitals may be input into the model training module with corresponding training data to create vitals models 735 that map any sensor measurement to the full range of possible heartrates 710, pulses 715, and/or other vitals. Thus all possible input sensor data may be mapped to a user's heartrate 710, pulse 715, and/or any other vital. The training visual data of a user's eye 705, training electrical data 720 and training optical data 725 may be collected during usage of a heartrate monitor distributed system. New training data may be collected during a usage of a heartrate monitor distributed system to periodically update the vitals models 735 and adapt the vitals models 735 to a user.

After the machine learning module 700 has been trained with the training heartrate 710, pulse 715, training visual data of user's eye 705, training electrical data 720, and/or training optical data 725, it produces vitals models 735 that may be used in machine learning module 750.

FIG. 7B is a block diagram of a second machine learning module 750 for determining a user's vitals, in accordance with an embodiment. Machine learning module 750 receives input measurements from any of the sensors located on eyewear devices and/or neckbands described herein, and uses the vitals models 735 created in module 700 to determine heartrate 770, pulse 775, and/or other vitals. Machine learning module 750 deals with a study of systems that can learn from data they are operating on, rather than follow only explicitly programmed instructions.

As shown in FIG. 7B, measured optical data 755, visual data of a user's eye 760, and/or electrical data 765 may be input to the vitals models 735 produced in machine learning module 700. In response, the vitals models 735 determines a likelihood that the measured optical data 755, visual data of a user's eye 760, and/or electrical data 765 corresponds to a particular heartrate 770, pulse 775, and/or other vitals. By combining optical data 755, visual data of a user's eye 760, and/or electrical data 765 all corresponding to one heartrate 770, pulse 775, and/or other vitals, the machine learning module 750 may improve the accuracy of the determine heartrate 770, pulse 775, and/or other vitals. Because electrical data 765 and optical data 765 are measured on non-traditional sections of a user's body, combining electrical data 765, optical data 765 and visual data of a user's eye 760 together may improve the accuracy of the determined heartrate 770, pulse 775 and/or other vitals. The adaptable nature of machine learning modules 700 and 750 to a particular user may also improve the accuracy of the determined heartrate 770, pulse 775 and/or other vitals.

Other vitals for which machine learning modules 700 and 750 may be used to determine may include blood pressure, blood-oxygen levels, body temperature, respiration rate, cardiac output, etc. Training electrical data 720 and electrical data 765 may be measured and provided to machine learning modules 700 and 750 by any of the electrical sensors described herein. Training optical data 725 and optical data 755 may be measured and provided to machine learning modules 700 and 750 by any of the optical sensors described herein. Training visual data of a user's eye 705 and visual data of a user's eye 760 may be measured and provided to machine learning modules 700 and 750 by a camera in the eye tracking system described with reference to FIG. 1, and/or any other camera located on any of the eyewear devices and/or neckbands described herein.

Machine learning modules 700 and 750 may be carried out by a processor located in the computation compartment 130 as described with reference to FIG. 1, and/or any other embedded processor in the eyewear devices described herein and/or a coupled computation device, such as a mobile device 815 as described in FIG. 8.

FIG. 8 is a block diagram of a heart rate monitor distributed system 800, in accordance with an embodiment. Heartrate monitor distributed system 800 includes an eyewear device 805, a neckband 810, and an optional mobile device 815. The eyewear device 805 may be the eyewear device as shown in FIG. 1-3A. The neckband 810 is connected to both the eyewear device 805 and the mobile device 815. The neckband 810 may be the neckband 135 as described in FIG. 1-2, 3B and 5. The neckband 810 may be the neckband 450 as described in FIG. 4. In alternative configurations of system 800, different and/or additional components may be included. The heartrate monitor distributed system 800 may operate in an adjusted reality system environment.

The eyewear device 805 includes optical systems 110, as described with reference to FIG. 1. The eyewear device 805 includes an optional eye tracker system 820 that collects visual data on the user's eye, one or more passive sensors 825, one or more active sensors 830, position sensors 835, and an Inertial Measurement Unit (IMU) 840. The eyewear device 805 includes electrical sensors 845 and optical sensors 850, as described in further detail with reference to FIG. 1-3A. As shown in FIG. 8, the eye tracker system 820 may be an optional feature of the eyewear device 805.

The eye tracker system 820 tracks a user's eye movement. The eye tracker system 820 may include at least a dichroic mirror, for reflecting light from an eye area towards a first position, and a camera at the position at which the light is reflected for capturing images. Based on the detected eye movement, the eye tracker system 820 may communicate with the neckband 810, CPU 865 and/or mobile device 815 for further processing. Eye tracking information collected by the eye tracker system 820 and processed by the CPU 865 of the neckband 810 and/or mobile device 815 may be used for a variety of display and interaction applications. The various applications include, but are not limited to, providing user interfaces (e.g., gaze-based selection), attention estimation (e.g., for user safety), gaze-contingent display modes (e.g., foveated rendering, varifocal optics, adaptive optical distortion correction, synthetic depth of field rendering), metric scaling for depth and parallax correction, etc. In some embodiments, a processor in the mobile device 815 may also provide computation for the eye tracker system 820, such as amplification of changes in visual information of a user's eye, as discussed with reference to FIG. 1.

Passive sensors 825 may be cameras. Passive sensors 825 may also be locators, which are objects located in specific positions on the eyewear device 805 relative to one another and relative to a specific reference point on the eyewear device 805. A locator may be a corner cube reflector, a reflective marker, a type of light source that contrasts with an environment in which the eyewear device 805 operates, or some combination thereof In embodiments in which the locators are active sensors 830 (i.e., an LED or other type of light emitting device), the locators may emit light in the visible band (˜370 nm to 750 nm), in the infrared (IR) band (˜750 nm to 1700 nm), in the ultraviolet band (300 nm to 380 nm), some other portion of the electromagnetic spectrum, or some combination thereof.

Based on the one or more measurement signals from the one or more position sensors 835, the IMU 840 generates IMU tracking data indicating an estimated position of the eyewear device 805 relative to an initial position of the eyewear device 805. For example, the position sensors 835 include multiple accelerometers to measure translational motion (forward/back, up/down, left/right) and/or multiple gyroscopes to measure rotational motion (e.g., pitch, yaw, and roll) and/or multiple magnetometers. In some embodiments, the IMU 840 rapidly samples the measurement signals and calculates the estimated position of the eyewear device 805 from the sampled data. For example, the IMU 840 integrates the measurement signals received from the accelerometers over time to estimate a velocity vector and integrates the velocity vector over time to determine an estimated position of a reference point of the eyewear device 805. Alternatively, the IMU 840 provides the sampled measurement signals to the neckband 810 and/or the mobile device 815 to process the computation to estimate the velocity vector and the estimated position of the eyewear device 805.

The IMU 840 may receive one or more calibration parameters from the neckband 810 and/or the mobile device 815. The one or more calibration parameters are used to maintain tracking of the eyewear device 805. Based on a received calibration parameter, the IMU 840 may adjust one or more IMU parameters (e.g., sample rate). The adjustment may be determined by the CPU 865 of the neckband 810, or a processor of the mobile device 815. In some embodiments, certain calibration parameters cause the IMU 840 to update an initial position of the reference point so it corresponds to a next calibrated position of the reference point. Updating the initial position of the reference point at the next calibrated position of the reference point helps reduce accumulated error associated with the determined estimated position of the eyewear device 805. The accumulated error, also referred to as drift error, causes the estimated position of the reference point to “drift” away from the actual position of the reference point over time. In some examples, the IMU 840 is located in the neckband 810 or an IMU is present in both the neckband 810 and eyewear device 805. In some examples, the IMU 840 receives position information from both position sensors 835 on the eyewear device 805 and position sensors 835 on the neckband (not shown).

The eyewear device includes electrical sensors 845, which may be located at positions on the eyewear device 805 in contact with a user's tissue. Electrical sensors 845 measure changes in an electrical potential associated with the systolic and diastolic stages of a user's cardiac cycle. An example of electrical data measured by electrical sensors 845 is shown in FIG. 6. There may be a plurality of electrical sensors 845 located on eyewear device 805. Electrical sensors 845 may provide electrical measurements to CPU 865 and/or mobile device 815. CPU 865 and/or mobile device 815 may calculate a user's vitals based on measurements provided by the electrical sensors 845. CPU 865 and/or mobile device 815 may calculate a user's vitals from electrical data using machine learning modules vitals models 735 and model training module 730 as discussed in FIG. 7A and 7B.

The eyewear device includes optical sensors 850, which may be located at positions on the eyewear device 805 in contact with a user's tissue. Optical sensors 850 measure changes in the absorption of a user's skin that result from volumetric changes associated with the systolic and diastolic stages of a user's cardiac cycle. An example of optical data measured by optical sensors 850 is shown in FIG. 6. There may be a plurality of optical sensors 850 located on the eyewear device 805. Optical sensors 850 may provide optical measurements to CPU 865 and/or mobile device 815. CPU 865 and/or mobile device 815 may calculate a user's vitals based on measurements provided by the optical sensors 850. CPU 865 and/or mobile device 815 may calculate a user's vitals from optical data using machine learning vitals models 735 and model training module 730 as discussed in FIG. 7A and 7B.

The neckband 810 includes a light source 855, power source 860, a CPU 865, light detectors 870, additional user vitals monitor 875, a wireless gateway 880, electrical sensors 885, activator 890, vitals models 735 and model training module 730. The additional user vitals monitor 875 and activator 890 may be optional components on the neckband 810. In some embodiments, the neckband 810 includes one or more multifunctional compartments that interface with various other optional functional units. Additional optional functional units can include, e.g., an audio unit, an additional power source, an additional processing unit (e.g., CPU), a projector, a reference camera, and the activator 890.

The light source 855 may be located on the neckband at a contact point with a user's tissue. Light source 855 may be light source 410 as shown in FIG. 4-5. The light source 855 may be optically coupled to the light detectors 870, such that the light source and light detectors 870 together produce an optical signal of a user's vitals. The light source may be a photodiode, LED, or any other device capable of emitting light.

The light detectors 870 may be located on the neckband at a contact point with a user's tissue. Light detectors 870 may be the light detectors 415 as shown in FIG. 4-5. The light detectors may be any photodetector, and may include bandpass filters tuned to the frequency of light emitted by the light source 855. The light detectors 870 may measure scattered light, reflected light and/or transmitted light through a user's tissue. Light detectors 870 may convey an optical measurement to the CPU 865, and/or machine learning modules vitals models 735 and model training module 730. Light detectors 870 may convey an optical measurement to any other embedded processor located in the eyewear device 805 and/or neckband 810 and/or mobile device 815. An example of an optical signal measured by light detectors 870 is shown in FIG. 6.

The power source 860 provides power to the optical systems 110, eye tracker system 820, passive sensors 825, active sensors 830, position sensors 835, IMU 840, electrical sensors 845 and optical sensors 850 on the eyewear device 805. The power source 860 provides power to the light source 855, CPU 865, light detectors 870, additional user vitals monitor 875, wireless gateway 880, electrical sensors 885 and activator 890 on the neckband 810. Power source 860 may be a rechargeable battery, which may be recharged by the mobile device 815. The power source 860 may be turned ON or OFF in response to a voice command detected by an optional audio unit, an input of the activator 890, and/or a command received by the mobile device 815.

The CPU 865 may be any standard processor, and may be the processor embedded in the computation compartment 130 as shown in FIG. 1-2 and FIG. 3B-5. The CPU 865 may provide all computational processing for the eyewear device 805, including the computation associated with the optical systems 110, eye tracker system 820, passive sensors 825, active sensors 830, IMU 840, electrical sensors 845 and/or optical sensors 850. The CPU 865 may carry out all computations associated with machine learning modules vitals models 735 and model training module 730. The CPU 865 may carry out calculations in parallel with the processor of the mobile device 815. A processor in the mobile device 815 may provide calculation results to the CPU 865.

The additional user vitals monitor 875 monitors additional vital signs and other user health indicators. Additional vital signs may be estimated calorie consumption, number of steps taken by the user, the user's temperature, respiration rate, blood pressure, etc. The additional user vitals monitor 875 may be located in close proximity to a user's neck on the neckband 810, so that the vital signs may be accurate. The additional user vitals monitor 875 may be thermally isolated or offset calibrated from the power source 860, light source 855 and CPU 865 to ensure that temperature estimates are a result of the user's temperature and are unaffected by heat generated by the power source 860, light source 855 and CPU 865. The additional user vitals monitor 875 may be in communication with the position sensors 835 and IMU 840 to detect user steps and user movement to estimate the number of steps taken and/or calorie consumption. Information measured by the additional user vitals monitor 875 may be conveyed to the CPU 865, vitals models 735, model training module 730 and/or mobile device 815, and may be used by the machine learning modules discussed with reference to FIG. 7A and 7B to estimate a user's vitals.

The wireless gateway 880 provides signal communication with the mobile device 815 and/or the eyewear device 805. The wireless gateway 880 may convey a signal from a wireless network to the mobile device 815 and/or to the neckband 810. The wireless gateway 880 may receive a signal from a wireless network from the mobile device 815. The wireless gateway 880 may be any standard wireless signal gateway, such as a Bluetooth gateway, Wi-Fi gateway, etc.

Electrical sensors 885 may be located at positions on the neckband 810 in contact with a user's tissue. Electrical sensors 885 measure changes in an electrical potential associated with the systolic and diastolic stages of a user's cardiac cycle. An example of electrical data measured by electrical sensors 885 is shown in FIG. 6. There may be a plurality of electrical sensors 885 located on neckband 810. Electrical sensors 885 may provide electrical measurements to CPU 865 and/or mobile device 815. CPU 865 and/or mobile device 815 may calculate a user's vitals based on measurements provided by the electrical sensors 885. CPU 865 and/or mobile device 815 may calculate a user's vitals from electrical data using machine learning modules vitals models 735 and model training module 730 as discussed in FIG. 7A and 7B.

The activator 890 controls functions on the neckband 810, the eyewear device 805, and/or the mobile device 815. The activator 890 may be an activation button located on the neckband 810. The activator 890 may power ON or OFF any of the units in the eyewear device 805 and/or neckband 810.

Machine learning modules located on the neckband 810 are the vitals models 735 and model training module 730. Vitals models 735 may be produced by the machine learning training module 730 from training data, and map measured signals to a user's vitals. The vitals models 735 are thus used to output a user's vitals from electrical signals measured by electrical sensors 845 and electrical sensors 885, optical signals measured by optical sensors 850 and light detectors 870, and visual data of a user's eye measured by the eye tracker system 820. Computation associated with the vitals models 735 and model training module 730 may be carried out by CPU 865 and/or mobile device 815. Vitals models 735 and model training module 730 may also input measurements made by the additional user vitals monitor 875 to determine a user's vitals. Vitals models 735 and model training module 730 are discussed in further detail with reference to FIG. 7A and 7B.

The heartrate monitor distributed system 800 determines a user's heartrate while also producing an AR, VR or MR environment for a user. The heartrate monitor distributed system 800 is able to adapt the experience of an AR, VR and/or MR environment based on a measurement of a user's heartrate. The heartrate monitor distributed system 800 is also able to distribute processing, sensing, power and heat generating functions across the eyewear device 805, neckband 810 and mobile device 815. This allows each of the eyewear device 805 and neckband 810 to be adjusted to the desired weight and temperature for user comfort, as well as providing varied virtual environment interfaces and functions for the user to interact with at any of the eyewear device 805, neckband 810 and/or mobile device 815.

### Additional Configuration Information

The foregoing description of the embodiments of the disclosure has been presented for the purpose of illustration; it is not intended to be exhaustive or to limit the disclosure to the precise forms disclosed. Persons skilled in the relevant art can appreciate that many modifications and variations are possible in light of the above disclosure.

Some portions of this description describe the embodiments of the disclosure in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the disclosure may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the disclosure may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosure of the embodiments is intended to be illustrative, but not limiting, of the scope of the disclosure, which is set forth in the following claim

What is claimed is:

1. A heartrate monitor device comprising:
a neckband;
an eyewear device communicatively coupled with the neckband, wherein at least one of the neckband and the eyewear device measures an electrical signal associated with a user's heart activity;
a light source optically coupled to a light detector, wherein the light source and the light detector are located on at least one of the neckband and the eyewear device, and measure an optical signal associated with the user's heart activity; and
a controller configured to determine a heartrate of the user based at least in part on the electrical signal and the optical signal.

2. The heartrate monitor device of claim 1, wherein:
the light source and the light detector are located on the neckband;
the light source transmits light through a portion of a user's neck; and
the optical signal is a measurement of an intensity of at least one of transmitted light, reflected light, and scattered light through the user's neck tissue.

3. The heartrate monitor device of claim 1, wherein:
the light source and the light detector are located on the eyewear device;
the light source transmits light through tissue of the user's ear; and
the optical signal is a measurement of an intensity of at least one of transmitted light, reflected light, and scattered light through tissue of the user's ear.

4. The heartrate monitor device of claim 1, wherein the eyewear device further comprises a nose pad secured to a user's nose, and wherein:
the light source and the light detector are located on the nose pad;
the light source transmits light through tissue of the user's nose; and
the optical signal is a measurement of an intensity of at least one of transmitted light, reflected light, and scattered light through tissue of the user's nose.

5. The heartrate monitor of claim 1, wherein the eyewear device further comprises a first electrode and a second electrode, and wherein the electrical signal associated with the user's heart activity is a voltage measured between the first electrode and the second electrode.

6. The heartrate monitor device of claim 1, wherein the eyewear device further comprises:
a first connector arm secured to a user's head;
a second connector arm secured to a user's head; and
wherein the electrical signal associated with the user's heart activity is a voltage measured between the eyewear device and at least one of the first connector arm and the second connector arm.

7. The heartrate monitor device of claim 1, wherein the neckband further comprises a first electrode secured to a user's neck and a second electrode secured to a user's neck, and wherein the electrical signal associated with the user's heart activity is a voltage measured between the first electrode and the second electrode.

8. The heartrate monitor device of claim 1, further comprising a camera configured to capture one or more images of a user's eye, and wherein the controller is configured to determine the heartrate of the user based at least in part on the visual information of the user's eye and surrounding tissue.

9. The heartrate monitor device of claim 1, wherein the controller further comprises:
a machine learning module comprising:
a model training module that generates a predictive model of the heartrate of the user based at least in part on one of: training electrical data, training optical data, training heartrate data, training pulse data, and training images of a user's eye.

10. The heartrate monitor device of claim 9, wherein the controller inputs the electrical signal and the optical signal into the predictive model of the heartrate of the user to determine the heartrate of the user.

11. The heartrate monitor device of claim 1, wherein the controller is located on the neckband, and the neckband provides power to the eyewear device.

12. A heartrate monitor device comprising:
a neckband;
a light source located on the neckband;
a light detector located on the neckband and optically coupled to the light source, wherein light is transmitted through a portion of the user's neck tissue and the light detector is configured to measure an optical signal;
a first electrode and a second electrode located on the neckband, wherein the first electrode and the second electrode are secured to the user's neck and configured to measure an electrical signal; and
a controller configured to determine a heartrate of the user based at least in part on the electrical signal and the optical signal.

13. The heartrate monitor device of claim 12, wherein the light source is at least an infrared light source, and the optical signal is an intensity of at least one of transmitted light, reflected light, and scattered light through tissue of the user's neck.

14. The heartrate monitor device of claim 12, wherein the neckband device is curved, and light is transmitted from the light source to the light detector through a segment of the neckband device curve.

15. The heartrate monitor device of claim 12, wherein multiple light detectors are located on the neckband device, and each light detector measures an intensity of transmitted light through a different segment of the neckband device curve.

16. The heartrate monitor device of claim 12, wherein the electrical signal is a voltage measured between the first electrode and the second electrode.

17. The heartrate monitor device of claim 12, further comprising:
a machine learning module comprising:
a model training module that generates a predictive model of the heartrate of the user based at least in part on one of: training electrical data, training optical data, training heartrate data, and training pulse data.

18. The heartrate monitor device of claim 17, wherein the controller inputs the electrical signal and the optical signal into the predictive model of the heartrate of the user to determine the heartrate of the user.

19. A heartrate monitor device comprising:
a neckband;
an eyewear device communicatively coupled with the neckband and configured to provide an adjusted reality environment to a user, wherein at least one of the neckband and the eyewear device measures a signal associated with the user's heart activity; and
a controller configured to determine a heartrate of the user based at least in part on the signal.

20. The heartrate monitor device of claim 19, wherein at least one of the neckband and the eyewear device further comprise:
a light detector optically coupled to the light source, wherein light is transmitted through a portion of the user's tissue and the light detector is configured to measure the signal.

21. The heartrate monitor device of claim 19, wherein at least one of the neckband and the eyewear device further comprise:
a first electrode and a second electrode, wherein the first electrode and the second electrode are secured to the user's tissue and are configured to measure the signal.
